(19) United States
(12) Patent Application Publication
(10) Pub. No.: US 2025/0134991 A1
(43) Pub. Date: May 1, 2025

(54) MONOPHOSPHORYL LIPID ADJUVANT (MPLA) COMPOSITIONS, VACCINE COMPOSITIONS THEREOF, AND METHODS OF PREPARING AND USING THE SAME

(71) Applicant: Thomas Jefferson University, Philadelphia, PA (US)

(72) Inventor: Matthias Johannes Schnell

(21) Appl. No.: 18/683,641

(22) PCT Filed: Aug. 19, 2022
(86) PCT No.: PCT/US22/40897

§ 371 Date: Feb. 14, 2024

Related U.S. Application Data
(60) Provisional application No. 63/235,348, filed on Aug. 20, 2021.

(51) Int. Cl. A61K 39/39; A61K 9/107; A61K 39/00; A61K 39/12; A61K 39/205; A61P 37/04

(52) U.S. Cl. CPC A61K 39/39; A61K 9/107; A61K 39/12; A61K 39/205; A61P 37/04; A61K 2039/55572

(57) ABSTRACT

The present invention relates, in part, to an oil-in-water emulsion comprising an emulsifying agent, an aqueous phase, an oil phase comprising squalene, and a monophosphoryl lipid adjuvant (MPLA), and methods of preparing and using the same. The present invention further relates to a vaccine composition comprising the emulsion of the present invention and an antigen and/or antigenic composition, and methods using the same.

### CROSS-REFERENCE TO RELATED APPLICATIONS

This application claims priority to U.S. Provisional Application No. 63/235,348 filed Aug. 20, 2021, which is hereby incorporated herein by reference in its entirety.

### STATEMENT REGARDING FEDERALLY SPONSORED RESEARCH

This invention was made with government support under HHSN272201700082C awarded by the National Institutes of Health. The government has certain rights in this invention.

### BACKGROUND OF THE INVENTION

Vaccines are widely used to treat and/or prevent viral and/or bacterial infections. Vaccines typically comprise an immunogenic antigen derived from a pathogen, or more recently a precursor thereof, which is detected by a host subject, resulting in the stimulation of an immune response in the host. The immunogenic antigen component of a vaccine may comprise inactivated virus (e.g. heat killed virus), attenuated live virus, and/or viral surface proteins (e.g., glycoproteins), among others. Ideally, the host immune system is exposed to a pathogenic antigen without the virulence associated with the pathogen, enabling the host to mount an immune response sufficient to eradicate the pathogen in the instance of a future exposure.

However, in many cases, the immune response stimulated upon exposure to an immunogenic antigen alone is of insufficient strength to offer protection upon future exposure to the pathogen. In such instances, an adjuvant may be administered in combination with the immunogenic antigen to improve the immune response, and thereby improve the efficacy of the vaccine.

There is thus a need in the art for compositions which elicit and/or enhance a desired antigen specific immune response in a subject, as well as methods of using such compositions. The present invention addresses this need.

### BRIEF SUMMARY OF THE INVENTION

The present invention provides, in part, an oil-in-water emulsion comprising an emulsifying agent, an aqueous phase, an oil phase comprising squalene, and a monophosphoryl lipid adjuvant (MPLA) of formula (I), wherein R¹, R², R³, R⁴, R⁵, and R⁶ are defined elsewhere

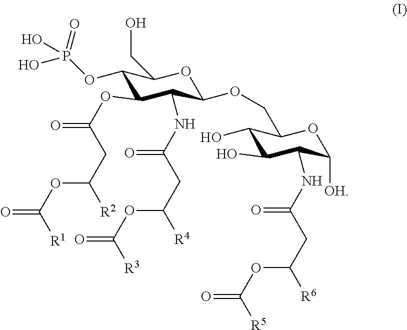

In certain embodiments, the aqueous phase comprises glycerol. In certain embodiments, the aqueous phase comprises an ammonium phosphate buffer.

In certain embodiments, the emulsifying agent comprises a non-ionic surfactant. In certain embodiments, the non-ionic surfactant comprises a polyoxyethylene-polyoxypropylene block copolymer.

In certain embodiments, the oil phase comprises a phospholipid. In certain embodiments, the phospholipid is 1,2-dimyristoyl-sn-glycero-3-phosphocholine (DMPC). In certain embodiments, the oil phase comprises (±)-α-tocopherol (vitamin E).

In certain embodiments, the emulsion comprises droplets. In certain embodiments, the droplets have a particle size ranging from about 70 to about 200 nm. In certain embodiments, the droplets have a polydispersity index (PDI) ranging from about 0.050 to about 0.150.

The present invention further provides a method for preparing the emulsion of the present invention, the method comprising:
stirring a mixture comprising the surfactant, the aqueous phase, and the oil phase to provide a first emulsion; and
homogenizing the first emulsion at an elevated pressure.

The present invention further provides a vaccine composition comprising an antigen and/or antigenic composition and the emulsion of the present invention. In certain embodiments, the antigen and/or antigenic composition comprises at least one viral vector.

In certain embodiments, the viral vector is derived from a virus selected from the group consisting of a coronavirus, filovirus, flavivirus, rhabdovirus, arenavirus, bunyavirus, poxvirus, paramyxovirus, orthomyxovirubs, Rabis virus (RABV), Lassa virus (LASV), Ebola virus (EBOV), Sudan virus (SUDV), Marburg virus, and influenza virus. In certain embodiments, the antigen and/or antigenic composition comprises at least one immunogenic polypeptide, or any fragment thereof. In certain embodiments, the at least one immunogenic polypeptide is derived from a virus selected from the group consisting of coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus. In certain embodiments, the immunogenic polypeptide is at least one selected from the group consisting of rabies glycoprotein (GP) antigen and Lassa glycoprotein complex (GPC) antigen.

In certain embodiments, the vaccine composition of the present disclosure comprises a rabies virus (RABV)-based vaccine vector, as previously described in the literature, including Blaney J E et al. (J. Virol, 2011, 85:10605-10616) and McGettigan J P et al. (J. Virol. 2003, 77:237-244), both of which are hereby incorporated by reference in their entireties. Thus, in certain embodiments, the vaccine composition of the present disclosure comprises an inactivated dual vaccine for LASV and RABV. This vaccine, named LASSARAB, expresses a codon-optimized version of LASV GPC (coGPC), alongside a modified RABV G, and was constructed upon a recombinant RABV recovery technology based on BNSP333 vector, a previously described highly attenuated RABV vaccine strain.

The present invention further provides a method of eliciting and/or enhancing a desired antigen specific immune response in a subject in need thereof, the method comprising administering to the subject the emulsion of the present invention or the vaccine composition of the present invention.

The present invention further provides a method of treating, preventing, and/or ameliorating an infection in a subject in need thereof, the method comprising administering to the subject the vaccine composition of the present invention. In certain embodiments, the infection is a viral infection. In certain embodiments, the viral infection is caused by at least one virus selected from the group consisting of coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus.

### BRIEF DESCRIPTION OF THE FIGURES

The drawings illustrate generally, by way of example, but not by way of limitation, various embodiments of the present application.

FIG. 1: illustrates the experimental timeline for immunization of C57BL/6 mice (n=5 per group). Mice were immunized in the gastrocnemius muscle with a total of 10 ug each of either LASSARAB or FILORAB1 inactivated viral particles adjuvanted with 5 μg of a TLR4 agonist in a 2% stable emulsion (PHAD-SE). All mice were primed on day 0 and boosted once on day 28, with sera collected on day 0, 21, and 28.

FIG. 2: Sera recovered from mice at day 21 post-immunization were diluted 1:50 and analyzed in a 3-fold serial dilution via an indirect ELISA to test for the relative quantities of EBOV GP-specific IgG antibodies. The delta values of OD₄₉₀ and OD₆₃₀ were compared to those for GP-specific monoclonal antibody 15H10 positive-control (star dotted line).

FIG. 3: Sera recovered from mice at day 28 post-immunization were diluted 1:50 and analyzed in a 3-fold serial dilution via an indirect ELISA to test for the relative quantities of EBOV GP-specific IgG antibodies. The delta values of OD₄₉₀ and OD₆₃₀ were compared to those for GP-specific monoclonal antibody 15H10 positive-control (star dotted line).

FIG. 4: EC50 values from total EBOV GP IgG ELISA curves were analyzed in FILORAB1 immunized mice. The results are presented as mean values at day 21 and 28.

FIG. 5: Sera recovered from FILORAB1-immunized mice at day 28 were diluted 1:50 and analyzed in a 3-fold serial dilution via an indirect ELISA to test for the relative quantities of EBOV GP-specific IgG2c and IgG1 antibodies. The delta values of OD₄₉₀ and OD₆₃₀ were compared to those of mouse sera from a previous experiment ME265 positive-control (star dotted-line).

FIG. 6: provides EC50 values from EBOV GP IgG2c and IgG1 ELISA curves which were analyzed in FILORAB1-immunized mice. A ratio of IgG2c and IgG1 isotype responses were assessed to evaluate the Th1-versus Th2 biased humoral immunity. The results are presented as mean values at day 28.

FIG. 7: provides a graph showing the particle size distribution of PHAD-SE (250 ug/mL).

FIG. 8: provides a graph showing the particle size distribution of PHAD-SE (75 ug/mL).

FIGS. 9A-9B: provide a graphical representation of a rabies virus vector (FIG. 9A) and LASSARAB vaccine construct (FIG. 9B).

FIG. 10: provides a schematic of the LASSARAB vaccine particle expressing Lassa GPC and Rabies GP at the particle surface.

FIG. 11: provides the chemical structure of PHAD, 3D-PHAD, and aPHAD.

FIGS. 12A-12B: provide graphs showing induction of Lassa GPC specific antibody titers elicited on vaccination with LASSARAB adjuvanted with either GLA-SE (FIG. 12A) or aPHAD-SE (FIG. 12B) as assessed by ELISA; x-axis: serum dilution; y-axis: optical density delta value; 9E9 is a monoclonal antibody directed against LASSA GPC and serves as a positive control.

FIGS. 13A-13C: provide bar graphs showing induction of Lassa GPC antibody isotypes elicited on vaccination with LASSARAB adjuvanted with either GLA-SE (FIG. 13A) or PHAD-SE (FIG. 13B), and a comparison of the ratio of Lassa GPC antibody isotypes (FIG. 13C).

FIG. 14: provides a graph showing survival of non-human primates (NHPs) after Lassa virus challenge of NHP immunized with LASSARAB+aPHAD-SE (treatment group) or Coravax+aPHAD-SE (control group); n=6/group.

FIG. 15: provides a graph showing serum viral load after Lassa virus challenge of NHPs immunized with either LASSARAB+aPHAD-SE (treatment group) or Coravax+aPHAD-SE (control group). Each individual NHP subject's viral load is graphed as a function of time.

FIGS. 16A-16D: provides images showing liver sections from LASSARAB and Coravax vaccinated NHPs. FIG. 16A N729 (Coravax vaccinated) liver; multifocal necrosis and loss of hepatocytes (circled) are visible which disrupt normal hepatic cord architecture. FIG. 16B: N726 (Coravax vaccinated) liver; necrosi and loss of much of the liver is observed in this section, with remaining hepatocytes individualized and free floating (arrows) in the necrotic debris. FIG. 16C: N863 (Coravax vaccinated) liver; hepatic artery is shown with severe arteritis; inflammatory cells are separating and unrveling the arterial wall (arrows). FIG. 16D: N797 (Lassarab vaccinated) liver; relatively normal section of liver is observed with intact hepatic cord architecture; portal veins (PV) have minimal lymphocytic inflammation surrounding them within the portal region which is commonly present in NHPs.

FIGS. 17A-17D: provide images showing heart sections from LASSARAB and Coravax vaccinated NHPs. FIG. 17A: shows that the epicardium and myocardial interstitium is expanded by inflammatory cells (arrows) composed of lymphocytes, macrophages, and neutrophils. Note the increased clear space between myocardiocytes (asterisks) indicating edema. The myocardiocytes are normal. FIG. 17B: N726 heart; shows that the endocardium is expanded by inflammatory cells, primarily lymphocytes. FIG. 17C: N797 heart; shows that normal epicardium (asterisk) and myocardium. FIG. 17D: N797 heart; shows normal endocardium (asterisk) and myocardium.

### DETAILED DESCRIPTION OF THE INVENTION

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

As used herein, each of the following terms has the meaning associated with it in this section. Unless defined otherwise, all technical and scientific terms used herein generally have the same meaning as commonly understood by one of ordinary skill in the art to which this invention belongs. Generally, the nomenclature used herein and the laboratory procedures in animal pharmacology, pharmaceutical science, separation science, and organic chemistry are those well-known and commonly employed in the art. It should be understood that the order of steps or order for performing certain actions is immaterial, so long as the present teachings remain operable. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

In the application, where an element or component is said to be included in and/or selected from a list of recited elements or components, it should be understood that the element or component can be any one of the recited elements or components and can be selected from a group consisting of two or more of the recited elements or components.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “adjuvant” as used herein refers to a substance that is capable of potentiating the immunogenicity of an antigen. Adjuvants can be one substance or a mixture of substances and function by acting directly on the immune system or by providing a slow release of an antigen.

Non-limiting examples of adjuvants include bacterial lipids, aluminum salts, polyanions, bacterial glycopeptides and/or polypeptides, and slow release agents such as Freund's incomplete adjuvant.

The term “ameliorating” or “treating” means that the clinical signs and/or the symptoms associated with a disease are lessened as a result of the actions performed. The signs or symptoms to be monitored will be well known to the skilled clinician.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

As used herein, the term “effective amount” or “therapeutically effective amount” means that amount of a composition (e.g., vaccine composition) or active ingredient (e.g., virus like particles (VLPs), virions, viral vectors, antigen, nucleic acid molecule) necessary to achieve an intended result e.g., to produce an intended immunological, pharmacological, therapeutic and/or protective result (e.g., that amount of VLPs, virions, or viral vectors sufficient to induce a measurable immune response, to prevent a particular disease condition, to reduce the severity of and/or ameliorate the disease condition or at least one symptom and/or condition associated therewith).

The terms “eliciting an immune response” or “immunizing” as used herein refer to the process of generating a B cell and/or a T cell response against a heterologous protein.

As used herein, the term “effective amount” or “therapeutically effective amount” means the amount of the virus like particle generated from vector of the invention which is required to prevent the particular disease condition, or which reduces the severity of and/or ameliorates the disease condition or at least one symptom thereof or condition associated therewith.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like. In certain embodiments, the present invention relates to vaccine compositions comprising immunogenic polypeptides (i.e., polypeptides possessing immunogenicity). In certain embodiments, the immunogenic polypeptide is any polypeptide derived from a virus selected from the group consisting of a coronavirus, filovirus, flavivirus, rhabdovirus, arenavirus, bunyavirus, poxvirus, paramyxovirus, orthomyxovirubs, Rabis virus (RABV), Lassa virus (LASV), Ebola virus (EBOV), Sudan virus (SUDV), Marburg virus, and influenza virus, which is suitable to elicit an immunogenic response, including any immunogenic polypeptides described in International Patent Applicant No. PCT/US2021/57743 and/or U.S. patent application Ser. No. 17/193,890, both of which are hereby incorporated by reference in their entireties.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

As used herein, the term “pharmaceutical composition” or “composition” refers to a mixture of at least one compound useful within the invention with a pharmaceutically acceptable carrier. The pharmaceutical composition facilitates administration of the compound to a subject.

As used herein, the term “pharmaceutically acceptable” refers to a material, such as a carrier or diluent, which does not abrogate the biological activity or properties of the compound useful within the invention, and is relatively non-toxic, i.e., the material may be administered to a subject without causing undesirable biological effects or interacting in a deleterious manner with any of the components of the composition in which it is contained.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the invention within or to the subject such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the subject. Supplementary active compounds may also be incorporated into the compositions. The “pharmaceutically acceptable carrier” may further include a pharmaceutically acceptable salt of the compound useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” refers to a salt of the administered compound prepared from pharmaceutically acceptable non-toxic acids and/or bases, including inorganic acids, inorganic bases, organic acids, inorganic bases, solvates (including hydrates) and clathrates thereof.

As used herein, a “pharmaceutically effective amount,” “therapeutically effective amount,” or “effective amount” of a compound is that amount of compound that is sufficient to provide a beneficial effect to the subject to which the compound is administered.

The term “prevent,” “preventing,” or “prevention” as used herein means avoiding or delaying the onset of symptoms associated with a disease or condition in a subject that has not developed such symptoms at the time the administering of an agent or compound commences. Disease, condition and disorder are used interchangeably herein.

A “subject” or “patient,” as used therein, may be a human or non-human mammal. Non-human mammals include, for example, livestock and pets, such as ovine, bovine, porcine, canine, feline and murine mammals. Preferably, the subject is human. In some embodiments, the subject is a domestic pet or livestock. In some embodiments, the subject is a cat. In some embodiments, the subject is a dog.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

“Titers” are numerical measures of the concentration of a virus or viral vector compared to a reference sample, where the concentration is determined either by the activity of the virus, or by measuring the number of viruses in a unit volume of buffer. The titer of viral stocks are determined, e.g., by measuring the infectivity of a solution or solutions (typically serial dilutions) of the viruses, e.g., on Hela cells using the soft agar method (see, Graham & Van Der eb (1973) Virology 52:456-467) or by monitoring resistance conferred to cells, e.g., G418 resistance encoded by the virus or vector, or by quantitating the viruses by UV spectrophotometry (see, Chardonnet & Dales (1970) Virology 40:462-477).

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term “treatment” therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “vaccination” as used herein refers to the process of inoculating a subject with an antigen to elicit an immune response in the subject, that helps to prevent or treat the disease or disorder the antigen is connected with. The term “immunization” is used interchangeably herein with vaccination.

A “vector” is a composition of matter which comprises a nucleic acid and which can be used to deliver the nucleic acid to the interior of a cell. Numerous vectors are known in the art including, but not limited to, linear polynucleotides, polynucleotides associated with ionic or amphiphilic compounds, plasmids, and viruses. In the present disclosure, the term “vector” includes an autonomously replicating virus. In certain embodiments, the viral vectors of the present invention can include any viral vector derived from a virus selected from the group consisting of a coronavirus, filovirus, flavivirus, rhabdovirus, arenavirus, bunyavirus, poxvirus, paramyxovirus, orthomyxovirubs, Rabis virus (RABV), Lassa virus (LASV), Ebola virus (EBOV), Sudan virus (SUDV), Marburg virus, and influenza virus. In certain embodiments, the viral vector can include a viral vector described in International Patent Applicant No. PCT/US2021/57743 and/or U.S. patent application Ser. No. 17/193,890, both of which are hereby incorporated by reference in their entireties.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compositions

In one aspect, the present invention provides an oil-in-water emulsion comprising an emulsifying agent, an aqueous phase, an oil phase comprising squalene, and a monophosphoryl lipid adjuvant (MPLA) of formula (I):

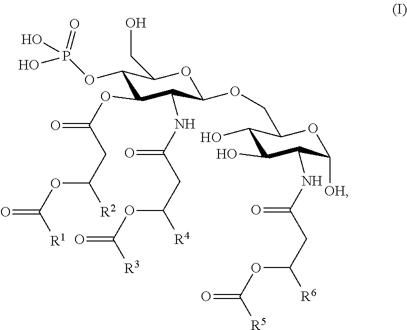

wherein:
each of R¹, R³, and R⁵ is tridecyl; and
each of R², R⁴, and R⁶ is undecyl;
   or a pharmaceutically acceptable salt, solvate, or stereoisomer thereof.

In certain embodiments, the salt comprises the ammonium salt thereof.

In certain embodiments, the compound of formula (I) is:

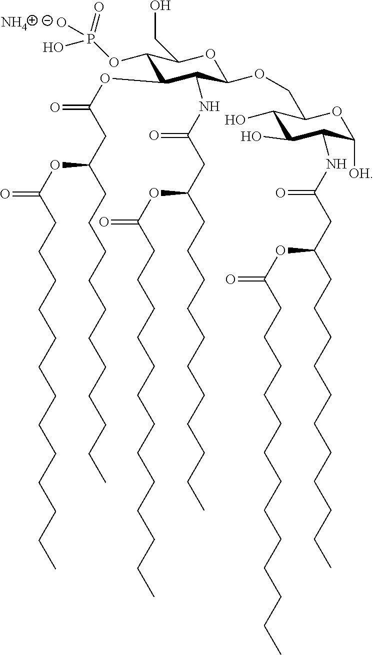

In certain embodiments, the MPLA has a concentration in the emulsion selected from the group consisting of about 1, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 405, 410, 415, 420, 425, 430, 435, 440, 445, 450, 455, 460, 465, 470, 475, 480, 485, 490, 495, and about 500 μg/mL.

In certain embodiments, the aqueous phase comprises glycerol. In certain embodiments, the glycerol has a concentration in the aqueous phase selected from the group consisting of 200, 205, 210, 215, 220, 225, 230, 235, 240, 245, 250, 255, 260, 265, 270, 275, 280, 285, 290, 295, and 300 mM. In certain embodiments, the concentration of glycerol in the aqueous phase is 258.4 mM.

In certain embodiments, the aqueous phase comprises an ammonium phosphate buffer. In certain embodiments, the ammonium phosphate buffer comprises (NH₄)H₂PO₄ and (NH₄)₂HPO₄, having a ratio selected from the group consisting of about 20:1, 19:1, 18:1, 17:1, 16:1, 15:1, 14:1, 13:1, 12:1, 11:1 and about 10:1 (w/w).

In certain embodiments, the ratio of (NH₄)H₂PO₄ and (NH₄)₂HPO₄ in the ammonium phosphate buffer is 16.5:1 (w/w), respectively.

In certain embodiments, the aqueous phase comprises a percentage of the emulsion selected from the group consisting of about 85, 86, 87, 88, 89, 90, 91, 92, 93, 94 and about 95% (v/v). In certain embodiments, the aqueous phase comprises about 92.4% (v/v) of the emulsion.

In certain embodiments, the emulsifying agent comprises a non-ionic surfactant. In certain embodiments, the non-ionic surfactant comprises a polyoxyethylene-polyoxypropylene block co-polymer. In certain embodiments, the polyoxyethylene-polyoxypropylene block co-polymer comprises a percentage of the emulsion selected from the group consisting of about 0.01, 0.02, 0.03, 0.04, 0.05, 0.06, 0.07, 0.08, 0.09, 0.10, 0.11, 0.12, 0.13, 0.14, and about 0.15% (w/v). In certain embodiments, the polyoxyethylene-polyoxypropylene block co-polymer comprises about 0.07% (w/v) of the emulsion.

In certain embodiments, the squalene comprises a percentage of the emulsion selected from the group consisting of about 1, 2, 3, 4, 5, 6, 7, 8, 9, 10, 11, 12, 13, 14 and about 15 (v/v). In certain embodiments, the squalene comprises about 8.55% (v/v) of the emulsion.

In certain embodiments, the oil phase comprises a phospholipid. In certain embodiments, the phospholipid is 1,2-dimyristoyl-sn-glycero-3-phosphocholine (DMPC). In certain embodiments, the DMPC comprises a percentage of the oil phase selected from the group consisting of about 10, 11, 12, 13, 14, 15, 16, 17, 18, 19, 20, 21, 22, 23, 24, 25, 26, 27, 28, 29, and about 30% (w/v). In certain embodiments, DMPC comprises about 19% (w/v) of the oil phase.

In certain embodiments, the oil phase comprises (±)-α-tocopherol (vitamin E). In certain embodiments, the (±)-α-tocopherol (vitamin E) comprises a percentage of the oil phase selected from the group consisting of about 0.1, 0.2, 0.3, 0.4, 0.5, 0.6, 0.7, 0.8, 0.9, and about 1.0% (w/v). In certain embodiments, the (±)-α-tocopherol (vitamin E) comprises about 0.5% (w/v) of the oil phase.

In certain embodiments, the emulsion comprises droplets. In certain embodiments, the droplets have a particle size selected from the group consisting of about 70, 75, 80, 85, 90, 95, 100, 105, 110, 115, 120, 125, 130, 135, 140, 145, 150, 155, 160, 165, 170, 175, 180, 185, 190, 195, and about 200 nm.

In certain embodiments, the droplets have a polydispersity index (PDI) selected from the group consisting of about 0.050, 0.055, 0.060, 0.065, 0.070, 0.075, 0.080, 0.085, 0.090, 0.095, 0.100, 0.105, 0.110, 0.115, 0.120, 0.125, 0.130, 0.135, 0.140, 0.145, and about 0.150.

In certain embodiments, the emulsion has a pH selected from the group consisting of about 5.5, 5.6, 5.7, 5.8, 5.9, 6.0, 6.1, 6.2, 6.3, 6.4, 6.5, 6.6, 6.7, 6.8, 6.9 and about 7.0. In certain embodiments, the pH is about 5.75.

In certain embodiments, the emulsion has a density selected from the group consisting of about 0.995, 0.996, 0.997, 0.998, 0.999, and about 1.0 g/mL.

In another aspect, the present invention provides a vaccine composition comprising an antigen and/or antigenic composition and the emulsion of the present invention. In certain embodiments, the antigen and/or antigenic composition comprises a viral vector. In certain embodiments, the viral vector is derived from a virus selected from a coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus. In certain embodiments, the antigen and/or antigenic composition comprises at least immunogenic polypeptide, or fragment thereof. In certain embodiments, the polypeptide comprises a protein. In certain embodiments, the polypeptide comprises a glycoprotein. In certain embodiments, the glycoprotein is a rabies virus glycoprotein (GP) antigen. In certain embodiments, the glycoprotein comprises a Lassa glycoprotein complex (GPC) antigen. In certain embodiments, the polypeptide, or any fragment thereof comprises a peptide (i.e., a peptide comprising 2 to 50 amino acids). In certain embodiments, the at least one immunogenic polypeptide is derived from a virus selected from a coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus.

In certain embodiments, the antigen and/or antigenic composition and the emulsion of the present invention have a weight ratio selected from the group consisting of about 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, and about 60:1.

In certain embodiments, squalene comprises about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, and about 4.0% of the vaccine composition (w/w). In certain embodiments, squalene comprises about 2.0% of the vaccine composition (w/w).

In certain embodiments, the present invention provides an oil-in-water emulsion comprising an emulsifying agent, an aqueous phase, an oil phase comprising squalene, and PHAD®. In certain embodiments, the present invention provides an oil-in-water emulsion comprising an emulsifying agent, an aqueous phase, an oil phase comprising squalene, and 3D-PHAD.

### Salts

The compounds described herein may form salts with acids or bases, and such salts are included in the present invention. The term “salts” embraces addition salts of free acids or bases that are useful within the methods of the invention. The term “pharmaceutically acceptable salt” refers to salts that possess toxicity profiles within a range that affords utility in pharmaceutical applications. In certain embodiments, the salts are pharmaceutically acceptable salts. Pharmaceutically unacceptable salts may nonetheless possess properties such as high crystallinity, which have utility in the practice of the present invention, such as for example utility in process of synthesis, purification or formulation of compounds useful within the methods of the invention.

Suitable pharmaceutically acceptable acid addition salts may be prepared from an inorganic acid or from an organic acid. Examples of inorganic acids include sulfate, hydrogen sulfate, hydrochloric, hydrobromic, hydriodic, nitric, carbonic, sulfuric, and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, fumaric, pyruvic, aspartic, glutamic, benzoic, anthranilic, 4-hydroxybenzoic, phenylacetic, mandelic, embonic (or pamoic), methanesulfonic, ethanesulfonic, benzenesulfonic, pantothenic, sulfanilic, 2-hydroxyethanesulfonic, trifluoromethanesulfonic, p-toluenesulfonic, cyclohexylaminosulfonic, stearic, alginic, β-hydroxybutyric, salicylic, galactaric, galacturonic acid, glycerophosphonic acids and saccharin (e.g., saccharinate, saccharate). Salts may be comprised of a fraction of one, one or more than one molar equivalent of acid or base with respect to any compound of the invention.

Suitable pharmaceutically acceptable base addition salts of compounds of the invention include, for example, ammonium salts and metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (or N-methylglucamine) and procaine. All of these salts may be prepared from the corresponding compound by reacting, for example, the appropriate acid or base with the compound.

### Methods

In one aspect, the present invention provides a method for preparing the emulsion of the present invention, the method comprising:
stirring a mixture comprising the surfactant, the aqueous phase, and the oil phase to provide a first emulsion; and
homogenizing the first emulsion at an elevated pressure.

In certain embodiments, the stirring occurs at a rate of about 8,000 rpm. In certain embodiments, the elevated pressure is about 30,000 psi. In certain embodiments, the emulsion is further subjected to filtration. In certain embodiments, the filtration comprises passage through at last one 0.22, 0.45, and/or 0.80 μm filter.

In another aspect, the present invention provides a method of eliciting and/or enhancing a desired antigen specific immune response in a subject in need thereof, comprising administering to the subject a pharmaceutically effective amount of the emulsion of the present invention or the vaccine composition of the present invention.

In another aspect, the present invention provides a method of treating, preventing, and/or ameliorating an infection in a subject in need thereof, comprising administering to the subject a pharmaceutically effective amount of the vaccine composition of the present invention. In certain embodiments, the infection is a viral infection. In certain embodiments, the viral infection is caused by at least one virus selected from the group consisting of coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus. When the viral infection is caused by a Coronavirus, the Coronavirus can be SARS-COV-2.

### Combination Therapies

In one aspect, the emulsion and/or vaccine compositions of the invention are useful within the methods of the invention in combination with one or more additional agents useful for treating viral infections. These additional agents may comprise compounds or compositions identified herein, or compounds (e.g., commercially available compounds) known to treat, prevent, or reduce the symptoms of viral infections, including but not limited to coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus infection. When the viral infection is caused by a Coronavirus, the Coronavirus can be SARS-COV-2.

### Kits

In one aspect, the present invention provides a kit for eliciting and/or enhancing a desired antigen specific immune response in a subject in need thereof, the kit comprising the emulsion of the present invention, and instructional materials for the use thereof.

In another aspect, the present invention provides a kit for eliciting and/or enhancing a desired antigen specific immune response in a subject in need thereof, the kit comprising the vaccine of the present invention, and instructional materials for the use thereof.

In another aspect, the present invention provides a kit for treating, preventing, and/or ameliorating a viral infection a subject in need thereof, comprising the vaccine composition of the present invention and instructional materials for the use thereof. In certain embodiments, the viral infection is caused by at least one virus selected from the group consisting of coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a patient, such as a mammal, such as a human, may be carried out using known procedures, at dosages and for periods of time effective to treat a disease or disorder contemplated herein. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a composition of the invention is from about 0.01 mg/kg to 100 mg/kg of body weight/per day. One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this invention may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the composition of the invention at levels lower than that required in order to achieve the desired therapeutic effect and gradually increase the dosage until the desired effect is achieved.

### Administration

Routes of administration of any of the compositions of the invention include inhalational, oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

### EXAMPLES

Various embodiments of the present application can be better understood by reference to the following Examples which are offered by way of illustration. The scope of the present application is not limited to the Examples given herein.

### Example 1: Preparation of Adjuvant Formulation

### Oil Phase Preparation

Vitamin E (342 mg) and squalene (58.69 g) were added to a Schott bottle, and the mixture was sonicated at 60° C. for 5 min. Next, 3D-(6-acyl)-PHAD® (60 mg), and subsequently 1,2-dimyristoyl-sn-glycero-3-phosphocholine (DMPC) (12.996 g), were added to the vessel comprising the vitamin E and squalene solution.

In certain embodiments, 20 mg of 3D-(6-acyl)-PHAD® was used. In certain embodiments, 200 mg of 3D-(6-acyl)-PHAD® was used.

The mixture was sonicated at 60° C. for 1-2 h, with gentle swirling of the vessel at each 30 min interval, until the mixture was fully dissolved.

### Aqueous Phase Preparation

To a beaker containing 800 g water for injection (WFI) was added (NH₄)H₂PO₄ (2.80 g), and the mixture was stirred until fully dissolved. Next, (NH₄)₂HPO₄ (170 mg) was added, and the mixture was stirred until completely dissolved to provide a buffered WFI solution. Glycerol (23.80 g) was subsequently added to the buffered WFI solution, with extensively rinsing and stirred until fully transferred and dissolved. Additionally, Pluronic F68 (547 mg) was added, and the mixture was stirred until completely dissolved.

### Emulsion Preparation

The oil phase was transferred from the Schott bottle to a vessel comprising the aqueous phase. The emulsion thus formed was mixed using a Silverson L5M mixer with ¾″ square hole high shear screen attached. The mixer head was lowered into the solution and the emulsion was mixed at 8,000 rpm for 15 minutes.

A 5 mL aliquot of the resultant emulsion was removed and subjected to dynamic light scattering (DLS) analysis. The remaining portion of the emulsion was transferred to a 1000 mL jacketed Schott bottle and chilled for 60 min.

Next, the emulsion was passed through a primed and WFI flushed M-110P microfluidizer. The emulsion was homogenized at 30,000 psi for 6 discrete passes and samples were collected after each set of two passes and subjected to DLS analysis. The particle size and polydispersity index (PDI) were measured using a Malvern Zetasizer NanoZSP and DLS methods.

The emulsion was subjected to further discrete passes through the microfluidizer until a particle size was obtained within the target range (i.e., 120±40 nm). The particle properties for discrete batches are provided in Tables 1-2.

TABLE 1
Homogenization of emulsion (75 μg/mL 3D-(6-acyl)-PHAD ®)

| Pass No. | Pressure (psi) | Duration (min) | Z-average (nm) | PDI |
|---|---|---|---|---|
| 0 | N/A | 15 | 257.5 | 0.439 |
| 2 | 30,000 | 7 | 179.3 | 0.106 |
| 4 | 30,000 | 7 | 138.9 | 0.074 |
| 6 | 30,000 | 7 | 121.5 | 0.092 |

TABLE 2
Homogenization of emulsion (250 μg/mL 3D-(6-acyl)-PHAD ®)

| Pass No. | Pressure (psi) | Duration (min) | Z-average (nm) | PDI |
|---|---|---|---|---|
| 0 | N/A | 15 | 578.4 | 0.489 |
| 2 | 30,000 | 7 | 177.5 | 0.115 |
| 4 | 30,000 | 7 | 142.6 | 0.074 |
| 6 | 30,000 | 7 | 120.3 | 0.078 |

In certain embodiments, the homogenized emulsions were further subjected to filtration. In certain embodiments, the filtration comprises an initial pass through a 0.45 μm filter, and two iterations of passage through 0.22 μm filters. In certain embodiments, the initial filtration comprises passage through a 0.80 μm filter.

### Example 2: Efficacy of PHAD-SE as an Adjuvant

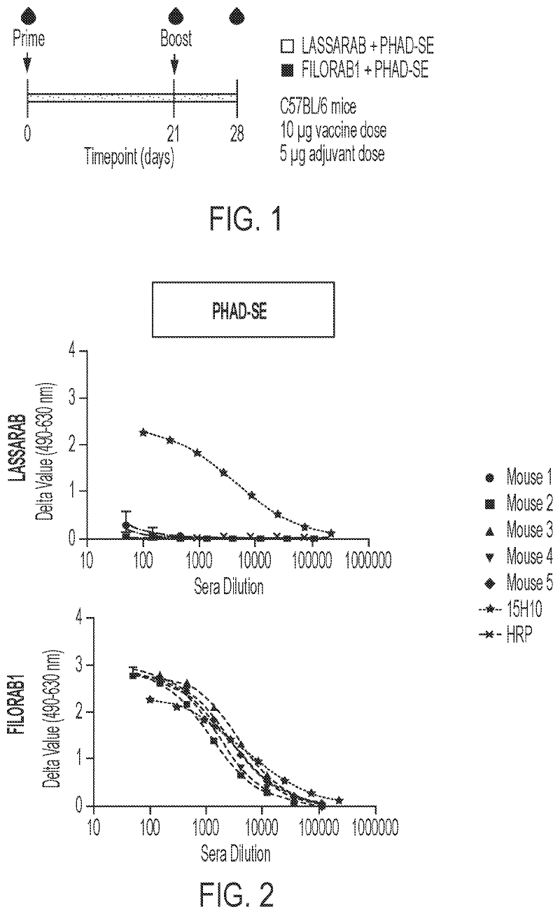

Mice were immunized in the gastrocnemius muscle with a total of 10 ug each of either LASSARAB or FILORAB1 inactivated viral particles adjuvanted with 5 μg of a TLR4 agonist in a 2% stable emulsion (PHAD-SE). All mice were primed on day 0 and boosted once on day 28, with sera collected on day 0, 21, and 28 (FIG. 1).

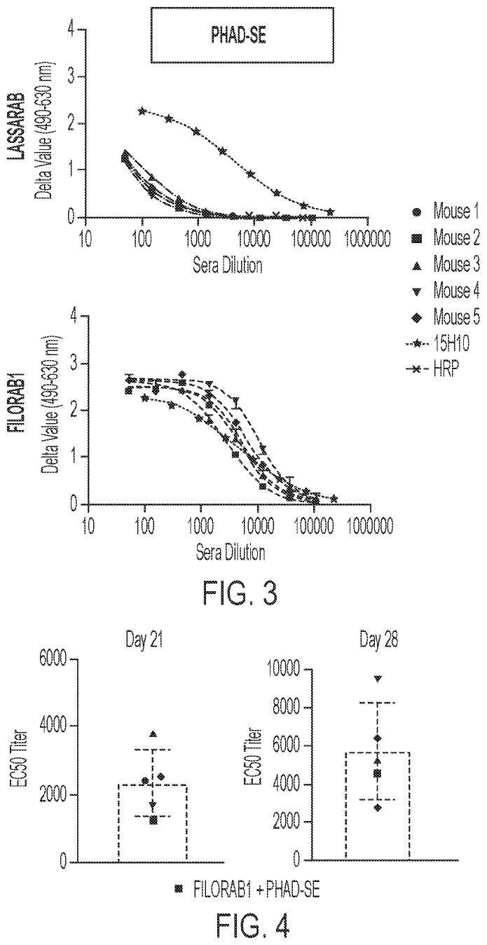

Sera was recovered from mice at day 21 and 28 post-immunization. Sera samples were diluted 1:50 and analyzed in a 3-fold serial dilution via an indirect ELISA to test for the relative quantities of EBOV GP-specific IgG antibodies. The delta values of OD₄₉₀ and OD₆₃₀ were compared to those for GP-specific monoclonal antibody 15H10 positive-control (FIGS. 2-3). EC50 values from total EBOV GP IgG ELISA curves were analyzed in FILORAB1 immunized mice (FIG. 4).

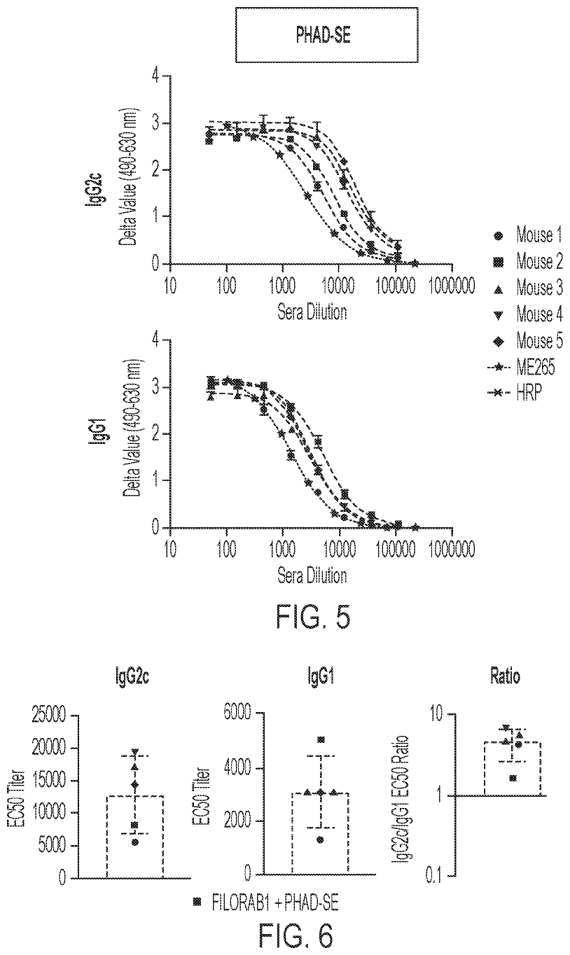

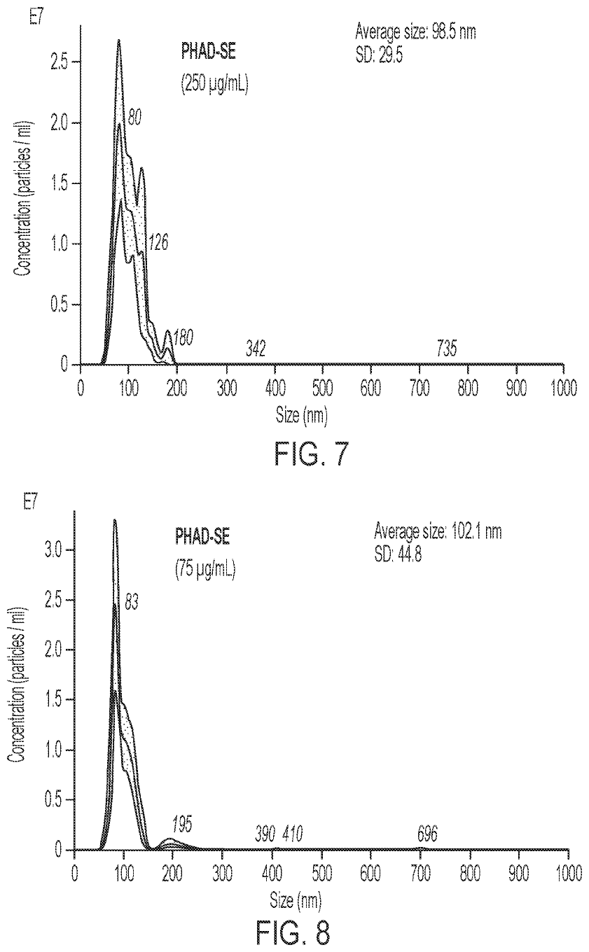

Sera recovered from FILORAB1-immunized mice at day 28 were diluted 1:50 and analyzed in a 3-fold serial dilution via an indirect ELISA to test for the relative quantities of EBOV GP-specific IgG2c and IgG1 antibodies. The delta values of OD 490 and OD₆₃₀ were compared to those of mouse sera from a previous experiment ME265 positive-control (FIG. 5).

EC50 values from EBOV GP IgG2c and IgG1 ELISA curves were analyzed in FILORAB1-immunized mice. A ratio of IgG2c and IgG1 isotype responses were assessed to evaluate the Th1-versus Th2 biased humoral immunity (FIG. 6).

### Example 3: LASSARAB+aPHAD-SE Vaccine Formulations

### Exemplary Generation and Recovery of Vaccine Vectors

To generate the vaccine vectors LASSARAB, LASSARABAG, and rVSV-GPC, the ORF of LASV GPC Josiah strain can be codon-optimized for mammalian transcription and synthetized by GenScript. LASV GPC can then be amplified by PCR and cloned between BsiWI and NheI restriction digest sites of BNSP333, thereby generating LASSARAB. LASSARABAG by removal of the native Rabies (RABV) glycoprotein (G) from the LASSARAB vector through the PacI and SmaI restriction digest sites and subsequent re-ligation with Klenow Fragment (Promega). rVSV-GPC can generated from VSV vector backbone from which the native VSV G was removed by MluI and NheI and can be further replaced by LASV GPC previously amplified by PCR to generate MluI and NheI restriction digest sites in the outward regions of 5′ and 3′ end of the ORF. Rhabdoviruses recovery can achieved by the same methods as described here. One skilled in the art appreciates that the disclosure is not limited to the exemplary species described herein, and that the vaccine vector generation and recovery methods described herein may be applied to additional embodiments not explicitly described herein.

### Exemplary Viral Production and Titer

LASSARAB, LASSARABAG, FILORAB1, rVSV-GPC, and SPBN viruses can be grown and titred according to methods known to those skilled in the art. Briefly, VERO-CCL81 cells can be inoculated with a multiplicity of infection (MOI) of 0.01 of each respective virus diluted in an appropriated volume of Serum Free media (Opti-Pro-Gibco). Briefly, viruses can be harvested up to a total of 4 times with media replacement (Opti-Pro) or until 80% cytopathic effect was detected. Tittering can be performed by limiting dilution focus forming assay and using FITC Rabies anti-N antibody stain. rVSV-GPC titers can be detected by plaque forming assay using a 2% methyl cellulose overlay.

### Exemplary Purification and Virus Inactivation

To produce inactivated LASSARAB and FILORAB1 vaccines, live viral supernatant can be sucrose purified and inactivated according to methods known to those skilled in the art. Briefly, viral supernatants can be concentrated at least 10 times and centrifuged at 110000 g through a 20% sucrose cushion. Virion pellets can be resuspended in PBS and B-propiolactone (BPL) can be added at a 1:2000 dilution for inactivation. Samples can be left at 4° C. O/N shaking and next day BPL hydrolysis can be conducted at 37° C. for 30 minutes.

### Lassa Virus

There are currently no approved vaccines for Lassa fever in clinical use. LASSARAB+aPHAD-SE is being developed as a vaccine for the prevention of Lassa fever. In non-limiting embodiments, the vaccine composition of the present invention is suitable for disease prevention in Lassa fever endemic areas. In additional non-limiting embodiments, the vaccine composition of the present invention is suitable for prevention of Lassa fever outbreaks and mitigation of disease spread, including spread by person-to-person contact or nosocomial spread. While the present invention describes embodiments of the vaccine composition for prevention of Lassa fever, LASSARAB+aPHAD-SE is suitable for additional indications, including but not limited to rabies and other endemic diseases.

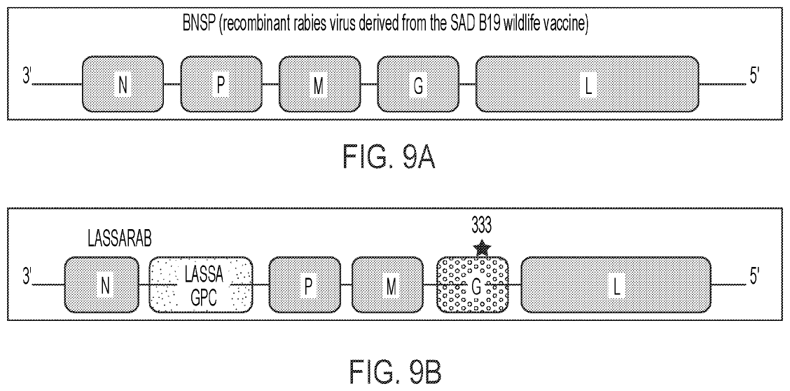

In certain embodiments, the antigen and/or antigenic composition comprises a Rabies-Vectored Lassa Fever Vaccine (LASSARAB) and a Phosphorylated Hexaacyl Disaccharides (PHAD-Stable squalene oil-in-water nanoemulsion (aPHAD-SE) adjuvant (LASSARAB+aPHAD-SE). In certain embodiments, the LASSARAB+aPHAD-SE is administered intramuscularly. In certain embodiments, the LASSARAB vaccine comprises a genetically modified rabies virus (RABV) vector, expressing both the rabies glycoprotein (GP) antigen and the full Lassa glycoprotein complex (GPC) antigen (FIGS. 9A-9B).

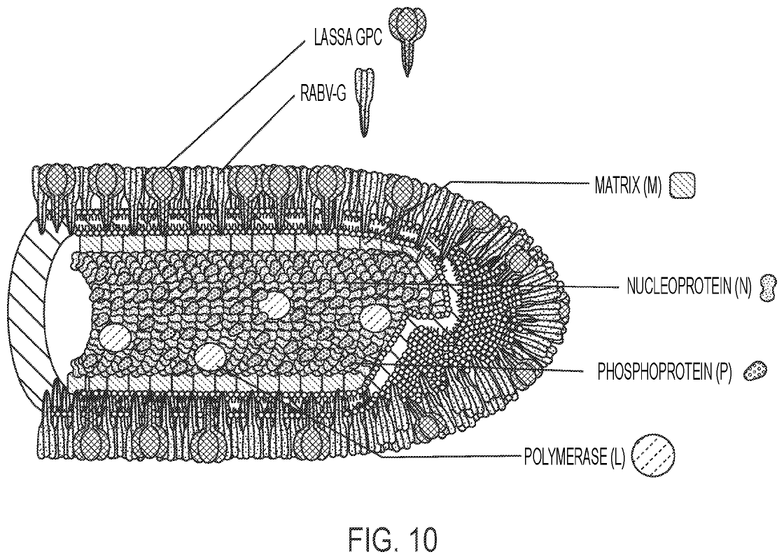

The parental RABV vaccine vector, BNSP, is a recombinant derivative of the SAD B19 RABV vaccine strain, which has been used as a live oral vaccine for wildlife in Europe. LASSARAB contains an Arg to Glu change at amino acid 333 of RABV G, which reduces viral neurotropism. Once the sequence is translated into a viral particle, it is then chemically inactivated with beta-propiolactone. The inactivated vaccine (FIG. 10) is delivered with a toll-like receptor 4 (TLR-4)-activating stable squalene oil-in-water nanoemulsion adjuvant, aPHAD-SE.

The aPHAD adjuvant is a fully synthetic form of monophosphoryl lipid A (MPLA), specifically 3D-(6-acyl) PHAD®. SE is a stabilized nanoemulsion of squalene and water. In certain embodiments, the final squalene ratio is 2% (volume/volume) in the LASSARAB+aPHAD-SE vaccine composition.

In certain embodiments, the LASSARAB vaccine is administered in combination with the aPHAD-SE adjuvant formulation in a two dose, prime-boost series (e.g., days 1 and 29). In certain embodiments, the LASSARAB vaccine is formulated as a liquid and is mixed with the aPHAD-SE formulation in the clinic pharmacy prior to administration by IM injection. Non-limiting formulations are provided in Table 3.

TABLE 3
Vaccine and adjuvant dose amounts and schedule

| Cohort | | | | Dosing |
|---|---|---|---|---|
| Dosage | Treatment Groups | Antigen | aPHAD-SE | Schedule |
| Low | LASSARAB + | 75 μg | 5 μg | D1, D29 |
| | aPHAD-SE | | | |
| Medium | LASSARAB + | 150 μg | 5 μg | D1, D29 |
| | aPHAD-SE | | | |
| High | LASSARAB + | 300 μg | 5 μg | D1, D29 |
| | aPHAD-SE | | | |

^{a}SE at 2% v/v for all doses

### Rabies Virus

95% of rabies disease burden is currently in Asia and Africa, and the incidence of rabies virus infection in humans in Africa is notably highest in West and East Africa. Thus, there is great need for more highly accessible rabies vaccines and adjusting features of rabies vaccines to enhance accessibility will limit the severe manifestations of this infection, with consequences at the individual and community level.

Utilizing LASSARAB in vaccination, the Rabies GP antigen is also delivered and is hypothesized to immunize against rabies, providing a platform with features potentially more favorable to accessible vaccination. In preclinical studies, LASSARAB adjuvanted with a TLR-4 agonist in stabilized emulsion (Glucopyranosyl lipid adjuvant in SE, GLA-SE), led to the generation of Lassa GPC specific antibodies as well as Rabies virus GP antibodies in nonhuman primates (NHP). The rabies titers elicited in NHP were several-fold above the titer of neutralization consistent with protection in humans.

LASSARAB administered to NHP with the adjuvant GLA-SE led to the induction of persistent high titer antibody responses to Lassa GPC as well as Rabies GP through at least 1 year post vaccination. Moreover, the NHPs were examined daily and no severe adverse events were noted over the full study period.

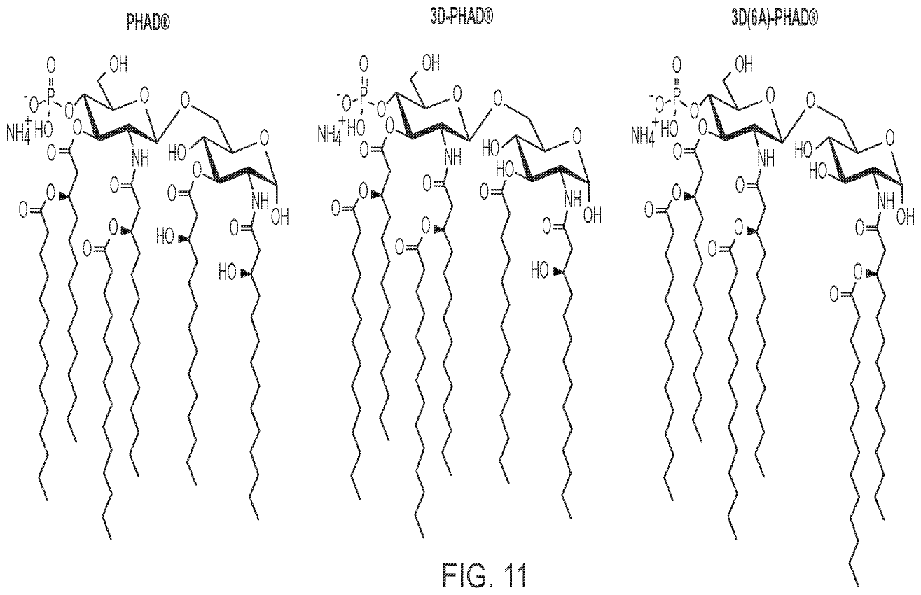

PHAD, synthesized by Avanti Lipids, is chemically identical to GLA. As with other synthetic MPLA analogs manufactured by Avanti, the PHAD molecules are each structurally homogeneous, highly purified, and mimic the TLR4 agonist activity of bacterial MPLA. The present invention, in one aspect, describes the use of aPHAD, specifically 3D-(6-Acyl) PHAD formulated in SE, as adjuvant. aPHAD is a highly purified chemically similar version of GLA. aPHAD, in comparison to PHAD, is more structurally similar to the MPLA used in the GlaxoSmithKline adjuvant systems such as AS01 and AS04. PHAD, 3D-PHAD and 3D-(6-Acyl) PHAD, referred to as aPHAD herein, independently or in formulation with other adjuvants, have been tested extensively in animals using a variety of antigens (FIG. 11). In these studies, these adjuvants exhibit a similar activity and safety profile to bacterially-derived MPLA.

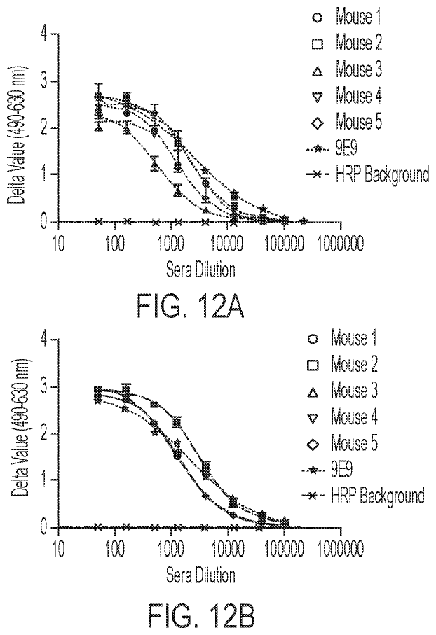

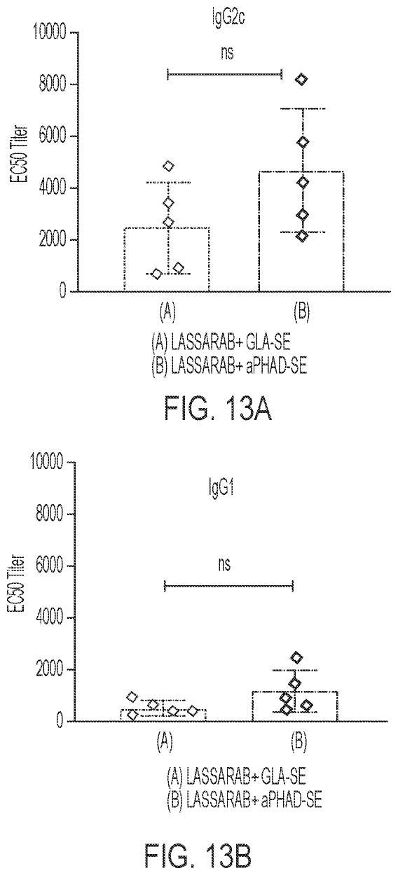

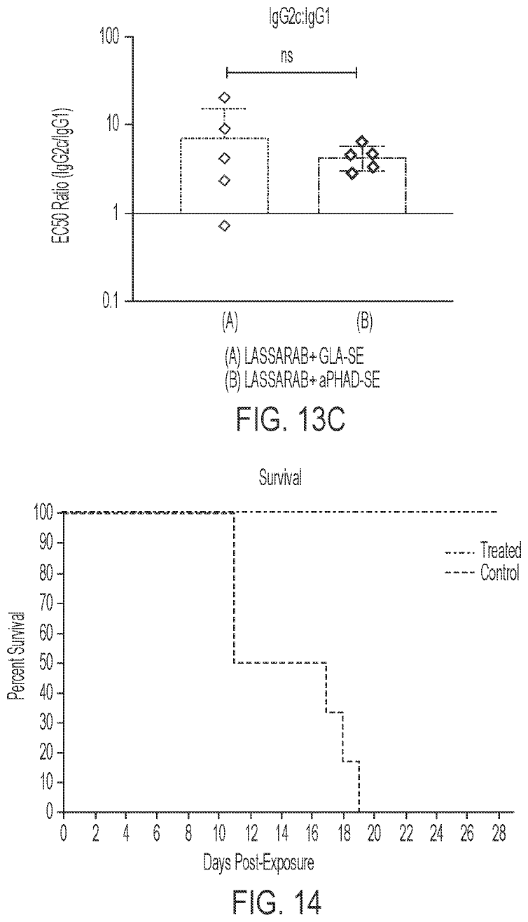

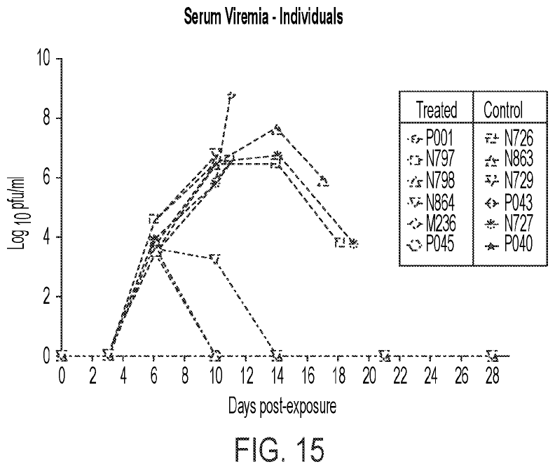

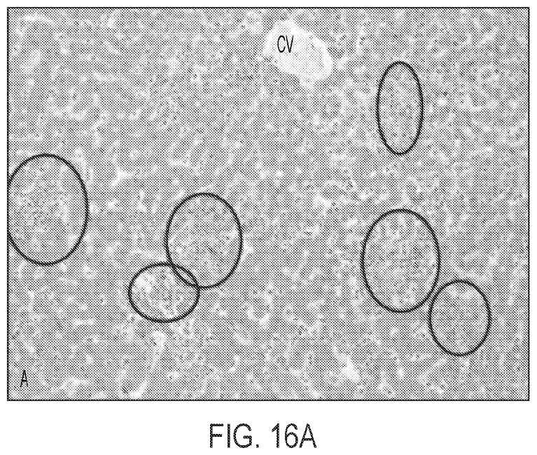

Bridging studies in which mice were immunized with LSSARAB adjuvant with either GLA-SE or aPHAD-SE showed no significant difference in the overall quantity of antigen-specific antibody responses (FIGS. 12A-12B and FIGS. 13A-13C).

NHPs that were vaccinated with LASSARAB+15 ug aPHAD-SE in 2% SE mounted robust antibody responses against the Lassa GPC and were protected after later challenge with Lassa virus, in contrast to control NHP receiving a different vaccine with identical adjuvant which succumbed to infection. In addition, no severe adverse effects of LASSARAB+aPHAD-SE were observed in NHPs over the period of observation of 70 days after vaccination and prior to challenge.

### Example 4: Primary Pharmacodynamics, Immunologic, and Efficacy Studies

Vaccine compositions comprising LASSARAB adjuvanted with a TLR-4 agonist in oil-in-water (GLA-SE) have been described in the art, as detailed in a Nature Communications publication by Abreu-Mota et al: “Non-neutralizing antibodies elicited by recombinant Lassa-Rabies vaccine are critical for protection against Lassa fever”. BNSP333 is a modified RABV vaccine strain (SAD B19) with an arginine-to-glutamate change at position 333 of RABV G that reduces neurotropism and improves its safety profile.

To generate LASSARAB, the LASV GPC sequence was inserted into the BNSP333 vector. The LASSARAB vector was expressed in Vero cells to generate the LASSARAB virion as previously described, wherein B-propiolactone was utilized to inactivate the LASSARAB virion. Sucrose-purified virions from infected Vero cells were analyzed by SDS-PAGE gel, Western blotting, and ELISA to confirm antigen expression. LASSARAB's potential as an inactivated vaccine depends on LASV GPC incorporation in LASSARAB-inactivated virions. LASV GP1/GP2 incorporation in LASSARAB was confirmed by Western blot analysis which demonstrated both GP1 (48-42 kDa) and GP2 (40-38 kDa) consistent with their respective molecular sizes.

On infection of Vero cells with LASSARAB, the expression of RABV and LASSA GPC proteins were identified at the cellular membrane by flow cytometry and immunofluorescence microscopy with antibodies directed against LASV GPC and/or RABV G.

### Rodent Studies

LASSARAB was found to be avirulent in mice. Swiss Webster mice were inoculated both intranasally (IN) and intraperitoneally (IP) with 106 foci-forming units (FFU) of LASSARAB, and animals were monitored for disease (e.g., hunched back and ruffled fur) and changes in weight for 28 days. IN exposure with BNSP (RABV group), which has been shown to be pathogenic after IN exposure, was used as a positive control, while PBS was used as negative control. On day 8, RABV-infected animals started to exhibit clinical signs of rabies, particularly weight loss. Mice inoculated with LASSARAB showed no clinical signs of disease.

The safety profile of the infectious LASSARAB vaccine was further characterized by intracranial inoculation (IC) in both adult BALB/c and adult severe combined immunodeficiency (SCID) mice. Increased pathogenicity was not observed following infections with LASSARAB compared with BNSP333 in either Balb/C or SCID mice. Finally, to confirm absent or decreased pathogenicity in a more sensitive animal model, Swiss Webster suckling mice were IC-exposed with LASSARAB or BNSP333. Independent of the virus dose used, LASSARAB or BNSP333 suckling mice started to succumb to the infection by day 7.

Antibody responses against LASV GPC are generated on immunization with LASSARAB, and titers are enhanced with addition of GLA-SE adjuvant. C57BL/6 mice were immunized IM in the gastrocnemius muscle with either 10 μg of B-propiolactone inactivated LASSARAB in PBS or adjuvanted with 5 μg of GLA, a TLR-4 agonist formulated in 2% of stable emulsion (SE); LASSARAB+GLA-SE, and boosted two times with the same amount on day 7 and 28. As early as day 28 post the initial prime vaccination there is observation of differences in the total IgG response against LASV GPC in the adjuvanted vs. unadjuvanted mice that received LASSARAB. By 42 days there is a further heightened enhancement of LASV GPC specific antibodies in the LASSARAB+GLA-SE vaccinated group compared to the mice receiving LASSARAB alone.

Additionally, LASSARAB vaccine efficacy was evaluated using outbred Hartley guinea pigs and the guinea pig-adapted LASV infection model. Six groups of 10 Hartley guinea pigs were used: 3 groups were immunized with inactivated LASSARAB+GLA-SE particles once (1), twice (2), or three times (3). All groups were challenged 58 days after the primary immunization with 10⁴ pfu of the guinea pig adapted LASV Josiah strain. The animals were monitored for viremia and clinical signs were recorded daily up to day 47 post-challenge. Significant protection was observed for animals immunized three times with LASSARAB+GLA-SE.

Furthermore, non-neutralizing LASV GPC-specific antibodies were found to be a major mechanism of protection by LASSARAB against Lassa fever through antibody-dependent cellular functions. Overall, these findings demonstrate an effective inactivated Lassa fever vaccine and elucidate a novel humoral correlate of protection for Lassa fever. Additionally, these data illustrate the importance of a TLR4 activating stable emulsion adjuvant in promoting optimal antibody responses for this vaccine.

### Non-Human Primate (NHP) Studies

TJU has studied the immune response generated by LASSARAB in an NHP model, as detailed in the publication by Kurup et al: “Tetravalent Rabies-Vectored Filovirus and Lassa Fever Vaccine Induces Long-term Immunity in Nonhuman Primates”. Here, NHP were immunized with LASSARAB+GLA-SE intramuscularly. The LASSARAB component was administered at a dose of 100 ug, with 15 μg of GLA-SE in 4% SE. The NHP were monitored daily and the vaccine-induced antibody response was characterized over time. LASV-GPC, and RABV-G IgG antibody responses were detected as early as 14 days after immunization. These antibodies were noted to persist through day 365, the duration of NHP observation. Peak LASV-GPC and RAB-V IgG responses were noted at day 56. RABV neutralizing titers were noted to be above the WHO-suggested protective threshold of 0.5 IU/mL on days 84 and 365.

To evaluate the protective efficacy of the LASSARAB vaccine, an NHP challenge study was performed. Twelve (12) nonhuman primates (NHPs) were randomly assigned into 2 groups of 6 NHPs each and vaccinated by IM administration with either 150 μg of LASSARAB or a control vaccine (i.e., CORAVAX), each adjuvanted with aPHAD-SE. CORAVAX consists of the BNSP vector (the same rabies vector as in LASSARAB), with Arg to Glu change at amino acid 333 of RABV G, and also contains an insertion encoding the SARS-COV-2 spike antigen, as described by Kurup et al. Both the LASSARAB vaccine and negative control vaccine CORAVAX were suspended in 15 ug adjuvant aPHAD-SE, with 2% SE formulation.

The NHPs were vaccinated on day 0 and day 28 with either LASSARAB+aPHAD-SE or CORAVAX+aPHAD-SE. Starting on day 0, the NHP were observed regularly for health and well-being. On Day 70, Each NHP was administered a target dose of 1000 PFU Lassa Virus via IM route. NHP were evaluated daily for changes in clinical signs. Rectal temperatures and weights were obtained, and physical exams performed on all scheduled blood collection days (Days 0, 3, 6, 10, 14, 21, and 28 after challenge), as well as on the day of euthanasia. Survival to overall study Day 98 (Day 28 post-challenge) was a primary endpoint. Animals judged moribund based on clinical signs and euthanasia criteria were euthanized. Blood samples were collected serially over the study to evaluate the animals by hematology, blood chemistries, serum antibody responses, and measurements of viremia. At necropsy, tissues were collected, preserved, processed, and examined microscopically for each NHP. All 6 of the NHP that were vaccinated with LASSARAB survived to the study endpoint, while all 6 NHP that were vaccinated with CORAVAX succumbed to severe disease (FIG. 14).

Only one of the LASSARAB-vaccinated NHPs developed clinical signs of illness, and these were considered mild and transient. In contrast, all the negative control NHP developed clinical signs of disease starting as early as Day 9 post-challenge, and these NHP succumbed to illness between days 10 and 18 Post-challenge (FIG. 14), consistent with historical data from the LASV-NHP challenge model. Post-challenge, the NHP receiving Coravax+aPHAD-SE had elevated white blood cell counts and neutrophils, and had marked transaminitis and hyperbilirubinemia, increased blood urea nitrogen, as well as loss of serum albumin, while the NHP receiving LASSARAB+aPHAD-SE had little changes in these lab tests compared to their group baseline.

NHPs receiving Coravax+aPHAD-SE had significantly higher viral titers in the serum relative to levels observed in the LASSARAB+aPHAD-SE group. LASSARAB+aPHAD-SE immunized NHP cleared the virus form the blood by day 19, while the Coravax+aPHAD-SE group had heightened viral load through time of death.

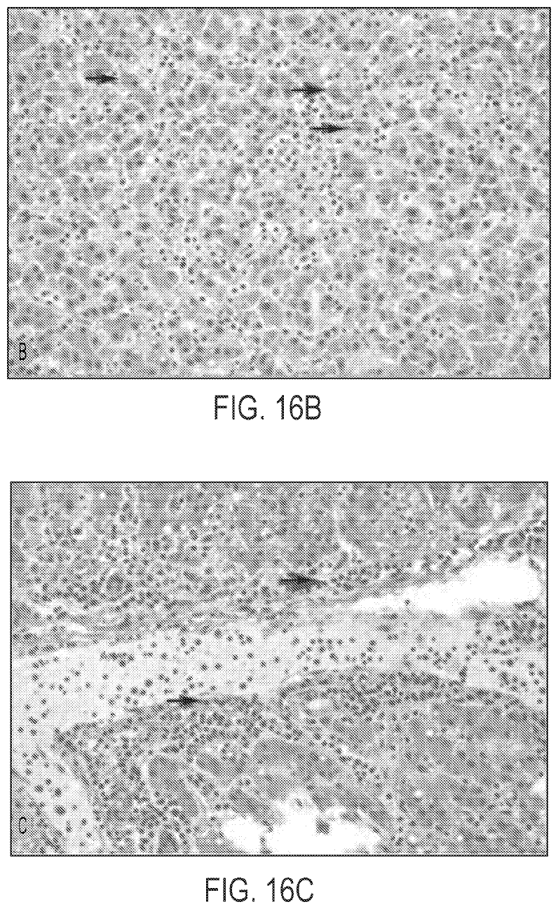

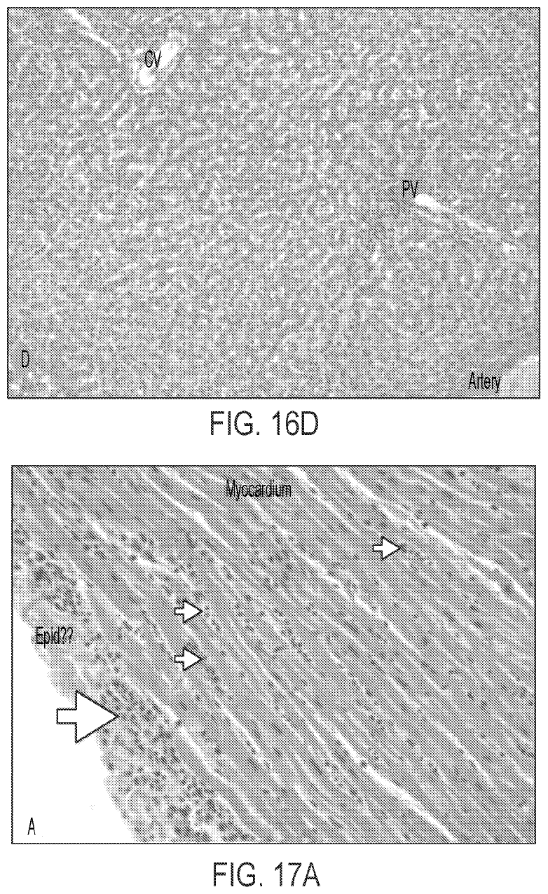

Features of disease at the organ and tissue level were assessed by a pathologist blinded to group in Coravax and LASSARAB vaccinated NHP after challenge. On gross examination, the lungs of 6/6 Coravax vaccinated NHP and 1/6 LASSARAB vaccinated NHP were described as red and non-collapsing; and the spleens of 6/6 Coravax vaccinated NHP and 0/6 LASSARAB vaccinated NHP were described as enlarged and friable. By histopathologic assessment there was evidence of necrotizing hepatitis in 5/6 of the Coravax vaccinated NHP and 0/6 of the LASSARAB vaccinated NHP (FIGS. 16A-16D). Alveolitis or interstitial pneumonitis was observed in 6/6 NHP that received Coravax while observed in 0/6 NHP that received LASSARAB.

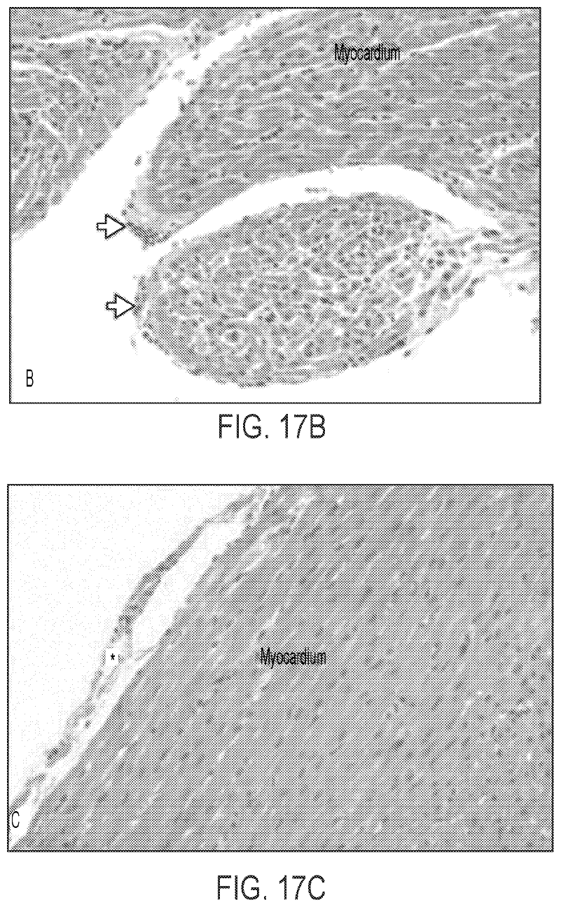

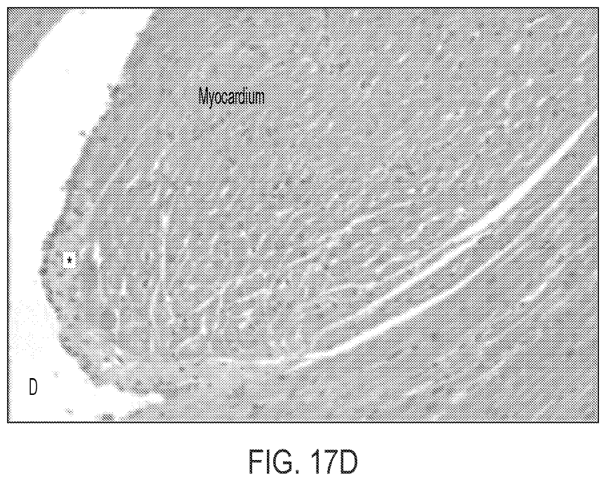

Additionally, inflammation in the choroid plexus, meninges, and heart muscle (FIGS. 17A-17D) was notably increased in the Coravax vaccinated NHP relative to the LASSARAB vaccinated NHP.

Of note, LASSARAB vaccinated NHP demonstrated evidence of chronic vascular changes at time of necropsy, described as similar in appearance to the human rheumatologic condition of Polyarteritis nodosa while Coravax vaccinated NHP demonstrated changes associated with acute vasculitis, associated with LASV infection. Inflammation of arterial vessels is a hallmark feature of the LASV challenge model and has previously been observed in unvaccinated NHP after challenge. Without wishing to be bound by theory, it is suspected that the qualities of inflammation differing between the Coravax and LASSARAB vaccinated NHP relate to time after the initial challenge, with surviving NHP demonstrating features of necrosis and repair, while those succumbing only manifest acute vascular changes.

### Enumerated Embodiments

The following exemplary embodiments are provided, the numbering of which is not to be construed as designating levels of importance:
Embodiment 1 provides an oil-in-water emulsion comprising an emulsifying agent, an aqueous phase, an oil phase comprising squalene, and a monophosphoryl lipid adjuvant (MPLA) of formula (I):

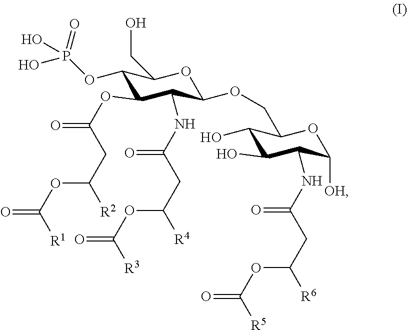

wherein:
R¹, R³, and R⁵ are tridecyl; and
R², R⁴, and R⁶ are undecyl;
   or a pharmaceutically acceptable salt, solvate, or stereoisomer thereof.
Embodiment 2 provides the emulsion of Embodiment 1, wherein the MPLA of formula (I) comprises:

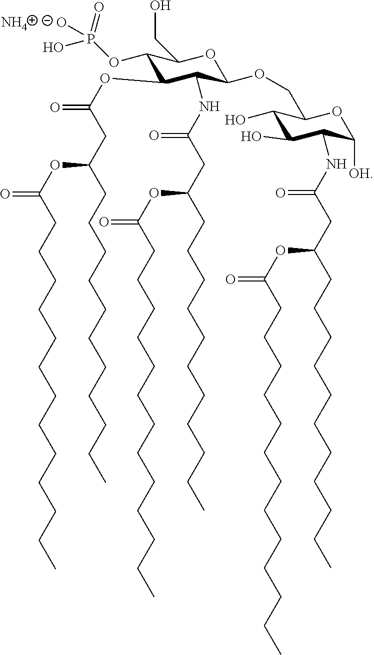

Embodiment 3 provides the emulsion of Embodiment 1 or 2, wherein the MPLA has a concentration in the emulsion ranging from about 1 μg/mL to about 500 μg/mL.
Embodiment 4 provides the emulsion of any one of Embodiments 1-3, wherein the concentration of MPLA is selected from the group consisting of about 5, about 25, about 50, about 75, and 250 μg/mL.
Embodiment 5 provides the emulsion of any one of Embodiments 1-4, wherein aqueous phase comprises glycerol.
Embodiment 6 provides the emulsion of Embodiment 5, wherein the glycerol has a concentration of about 200 to about 300 mM in the aqueous phase.
Embodiment 7 provides the emulsion of any one of Embodiments 1-6, wherein the aqueous phase comprises an ammonium phosphate buffer.
Embodiment 8 provides the emulsion of any one of Embodiments 1-7, wherein the ammonium phosphate buffer comprises (NH₄)H₂PO₄ and (NH₄)₂HPO₄, having a ratio of about 16.5:1 (w/w), respectively.
Embodiment 9 provides the emulsion of any one of Embodiments 1-8, wherein the aqueous phase comprises about 85% to about 95% (v/v) of the emulsion.
Embodiment 10 provides the emulsion of Embodiment 9, wherein the aqueous phase comprises about 92.4% (v/v) of the emulsion.
Embodiment 11 provides the emulsion of any one of Embodiments 1-10, wherein the emulsifying agent comprises a non-ionic surfactant.
Embodiment 12 provides the emulsion of Embodiment 11, wherein the non-ionic surfactant comprises a polyoxyethylene-polyoxypropylene block co-polymer.
Embodiment 13 provides the emulsion of Embodiment 12, wherein the polyoxyethylene-polyoxypropylene block co-polymer comprises about 0.01% to about 0.15% (w/v) of the emulsion.
Embodiment 14 provides the emulsion of Embodiment 13, wherein the polyoxyethylene-polyoxypropylene block co-polymer comprises about 0.07% (w/v) of the emulsion.
Embodiment 15 provides the emulsion of any one of Embodiments 1-14, wherein the squalene comprises about 1% to about 15% (v/v) of the emulsion.
Embodiment 16 provides the emulsion of Embodiment 15, wherein the squalene comprises about 8.55% (v/v) of the emulsion.
Embodiment 17 provides the emulsion of any one of Embodiments 1-16, wherein the oil phase comprises a phospholipid.
Embodiment 18 provides the emulsion of Embodiment 17, wherein the phospholipid is 1,2-dimyristoyl-sn-glycero-3-phosphocholine (DMPC).
Embodiment 19 provides the emulsion of Embodiment 18, wherein the DMPC comprises about 10% to about 30% (w/v) of the oil phase.
Embodiment 20 provides the emulsion of Embodiment 19, wherein the DMPC comprises about 19% (w/v) of the oil phase.
Embodiment 21 provides the emulsion of any one of Embodiments 1-20, wherein the oil phase comprises (±)-α-tocopherol (vitamin E).
Embodiment 22 provides the emulsion of Embodiment 21, wherein the (±)-α-tocopherol (vitamin E) comprises about 0.1% to about 1.0% (w/v) of the oil phase.
Embodiment 23 provides the emulsion of Embodiment 22, wherein the (±)-α-tocopherol (vitamin E) comprises about 0.5% (w/v) of the oil phase.
Embodiment 24 provides the emulsion of any one of Embodiments 1-23, wherein the emulsion comprises droplets.
Embodiment 25 provides the emulsion of Embodiment 24, wherein the droplets have a particle size ranging from about 70 to about 200 nm.
Embodiment 26 provides the emulsion of Embodiment 24 or 25, wherein the droplets have a polydispersity index (PDI) ranging from about 0.050 to about 0.150.
Embodiment 27 provides the emulsion of any one of Embodiments 1-26, wherein the emulsion has a pH ranging from about 5.5 to about 6.0.
Embodiment 28 provides the emulsion of any one of Embodiments 1-27, wherein the emulsion has a density of about 1.0 g/mL.
Embodiment 29 provides a method for preparing the emulsion of Embodiment 1, the method comprising:
stirring a mixture comprising the surfactant, the aqueous phase, and the oil phase to provide a first emulsion; and
homogenizing the first emulsion at an elevated pressure.
Embodiment 30 provides the method of Embodiment 29, wherein the stirring occurs at a rate of about 8,000 rpm.
Embodiment 31 provides the method of Embodiment 29 or 30, wherein the elevated pressure is about 30,000 psi.
Embodiment 32 provides a vaccine composition comprising an antigen and/or antigenic composition and the emulsion of any one of Embodiments 1-28.
Embodiment 33 provides the vaccine composition of Embodiment 32, wherein the antigen and/or antigenic composition comprises at least one viral vector.
Embodiment 34 provides the vaccine composition of Embodiment 33, wherein the viral vector is derived from a virus selected from the group consisting of a coronavirus, filovirus, flavivirus, rhabdovirus, arenavirus, bunyavirus, poxvirus, paramyxovirus, orthomyxovirubs, Rabis virus (RABV), Lassa virus (LASV), Ebola virus (EBOV), Sudan virus (SUDV), Marburg virus, and influenza virus.
Embodiment 35 provides the vaccine composition of Embodiment 32, wherein the antigen and/or antigenic composition comprises at least one immunogenic polypeptide, or any fragment thereof.
Embodiment 36 provides the vaccine composition of Embodiment 35, wherein the at least one immunogenic polypeptide is derived from a virus selected from the group consisting of coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus.
Embodiment 37 provides the vaccine composition of Embodiment 36, wherein the immunogenic polypeptide is at least one selected from the group consisting of rabies glycoprotein (GP) antigen and Lassa glycoprotein complex (GPC) antigen.
Embodiment 38 provides the vaccine composition of Embodiment 32-37, wherein the antigen and/or antigenic composition and the emulsion of any one of Embodiments 1-28 have a weight ratio of about 15:1 to about 60:1 (w/w).
Embodiment 39 provides the vaccine composition of Embodiment 32-38, wherein the squalene in the emulsion of any one of Embodiments 1-28 comprises about 2% (v/v) of the vaccine composition.
Embodiment 40 provides a method of eliciting and/or enhancing a desired antigen specific immune response in a subject in need thereof, comprising administering to the subject the emulsion of any one of Embodiments 1-28 or the vaccine composition of any one of Embodiments 32-39.
Embodiment 41 provides a method of treating, preventing, and/or ameliorating an infection in a subject in need thereof, comprising administering to the subject the vaccine composition of any one of Embodiments 32-39.
Embodiment 42 provides the method of Embodiment 41, wherein the infection is a viral infection.
Embodiment 43 provides the method of Embodiment 42, wherein the viral infection is caused by at least one virus selected from the group consisting of coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus.

The terms and expressions employed herein are used as terms of description and not of limitation, and there is no intention in the use of such terms and expressions of excluding any equivalents of the features shown and described or portions thereof, but it is recognized that various modifications are possible within the scope of the embodiments of the present application. Thus, it should be understood that although the present application describes specific embodiments and optional features, modification and variation of the compositions, methods, and concepts herein disclosed may be resorted to by those of ordinary skill in the art, and that such modifications and variations are considered to be within the scope of embodiments of the present application.

1. An oil-in-water emulsion comprising an emulsifying agent, an aqueous phase, an oil phase comprising squalene, and a monophosphoryl lipid adjuvant (MPLA) of formula (I): 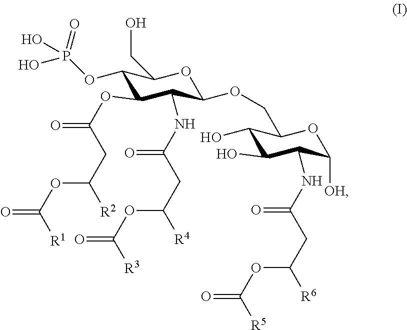
wherein:
R¹, R³, and R⁵ are tridecyl; and
R², R⁴, and R⁶ are undecyl;
or a pharmaceutically acceptable salt, solvate, or stereoisomer thereof.

2. The emulsion of claim 1, wherein the MPLA of formula (I) comprises:

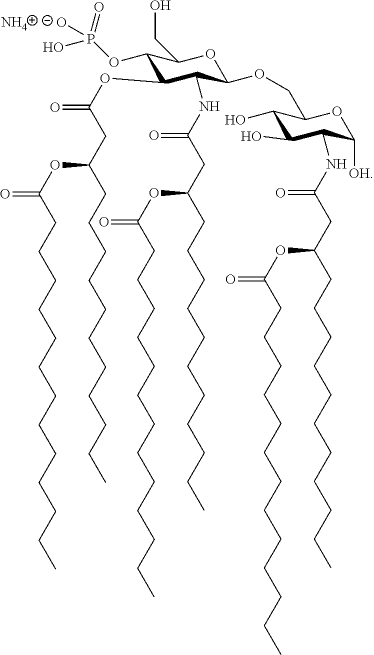

3. The emulsion of claim or 2, wherein at least one of the following applies:
(a) the MPLA has a concentration in the emulsion ranging from about 1 μg/mL to about 500 μg/mL;
(b) the squalene comprises about 1% to about 15% (v/v) of the emulsion, optionally wherein the squalene comprises about 8.55% (v/v) of the emulsion;
(c) the aqueous phase comprises glycerol;
(d) the aqueous phase comprises an ammonium phosphate buffer;
(e) the aqueous phase comprises about 85% to about 95% (v/v) of the emulsion;
(f) the emulsifying agent comprises a non-ionic surfactant;
(g) the oil phase comprises a phospholipid; and
(h) the oil phase comprises a-tocopherol (vitamin E).

4. The emulsion of claim 3, wherein at least one of the following applies:
(a) the concentration of MPLA is selected from the group consisting of about 5, about 25, about 50, about 75, and 250 μg/mL;
(b) the glycerol has a concentration of about 200 to about 300 mM in the aqueous phase;
(c) the ammonium phosphate buffer comprises (NH₄)H₂PO₄ and (NH₄)₂HPO₄, having a ratio of about 16.5:1 (w/w), respectively;
(d) the aqueous phase comprises about 92.4% (v/v) of the emulsion;
(e) the phospholipid is 1,2-dimyristoyl-sn-glycero-3-phosphocholine (DMPC);
(f) the droplets have a particle size ranging from about 70 to about 200 nm; and
(g) the droplets have a polydispersity index (PDI) ranging from about 0.050 to about 0.150.

5-11. (canceled)

12. The emulsion of claim 3, wherein the non-ionic surfactant comprises a polyoxyethylene-polyoxypropylene block co-polymer.

13. The emulsion of claim 12, wherein the polyoxyethylene-polyoxypropylene block co-polymer comprises about 0.01% to about 0.15% (w/v) of the emulsion, optionally wherein the polyoxyethylene-polyoxypropylene block co-polymer comprises about 0.07% (w/v) of the emulsion.

14-18. (canceled)

19. The emulsion of claim 4, wherein the DMPC comprises about 10% to about 30% (w/v) of the oil phase, optionally wherein the DMPC comprises about 19% (w/v) of the oil phase.

20-21. (canceled)

22. The emulsion of claim 3, wherein the (±)-α-tocopherol (vitamin E) comprises about 0.1% to about 1.0% (w/v) of the oil phase, optionally wherein the (±)-α-tocopherol (vitamin E) comprises about 0.5% (w/v) of the oil phase.

23. (canceled)

24. The emulsion of claim 1, wherein the emulsion comprises droplets.

25. The emulsion of claim 24, wherein at least one of the following applies:
(a) the droplets have a particle size ranging from about 70 to about 200 nm; and
(b) the droplets have a polydispersity index (PDI) ranging from about 0.050 to about 0.150.

26. (canceled)

27. The emulsion of claim 1, wherein at least one of the following applies:
(a) the emulsion has a pH ranging from about 5.5 to about 6.0; and
(b) the emulsion has a density of about 1.0 g/mL.

28. (canceled)

29. A method for preparing the emulsion of claim 1, the method comprising:
stirring a mixture comprising the surfactant, the aqueous phase, and the oil phase to provide a first emulsion; and
homogenizing the first emulsion at an elevated pressure.

30. The method of claim 29, wherein at least one of the following applies:
(a) the stirring occurs at a rate of about 8,000 rpm; and
(b) the elevated pressure is about 30,000 psi.

31. (canceled)

32. A vaccine composition comprising an antigen and/or antigenic composition and the emulsion of claim 1, optionally wherein the antigen and/or antigenic composition comprises at least one viral vector or at least one immunogenic polypeptide, or any fragment thereof.

33. (canceled)

34. The vaccine composition of claim 32, wherein the viral vector or immunogenic polypeptide is derived from a virus selected from the group consisting of a coronavirus, filovirus, flavivirus, rhabdovirus, arenavirus, bunyavirus, poxvirus, paramyxovirus, orthomyxovirus, Rabis virus (RABV), Lassa virus (LASV), Ebola virus (EBOV), Sudan virus (SUDV), Marburg virus, and influenza virus.

35-36. (canceled)

37. The vaccine composition of claim 32, wherein the immunogenic polypeptide is at least one selected from the group consisting of rabies glycoprotein (GP) antigen and Lassa glycoprotein complex (GPC) antigen.

38. The vaccine composition of claim 32, wherein at least one of the following applies:
(a) the antigen and/or antigenic composition and the emulsion have a weight ratio of about 15:1 to about 60:1 (w/w); and
(b) the squalene in the emulsion comprises about 2% (v/v) of the vaccine composition.

39. (canceled)

40. A method of eliciting and/or enhancing a desired antigen specific immune response in a subject in need thereof, comprising administering to the subject the emulsion of claim 1, or a vaccine composition thereof comprising an antigen and/or antigenic composition.

41. A method of treating, preventing, and/or ameliorating an infection in a subject in need thereof, comprising administering to the subject the vaccine composition of claim 32.

42. The method of claim 41, wherein the infection is a viral infection, optionally wherein the viral infection is caused by at least one virus selected from the group consisting of coronavirus, filovirus, flavivirus, rhabdovirus, arenavirus, bunyavirus, poxvirus, paramyxovirus, orthomyxovirus, Rabies virus (RABV), Lassa virus (LASV), Ebola virus (EBOV), Sudan virus (SUDV), Marburg virus, and influenza virus.

43. (canceled)
